(19) United States
(12) Patent Application Publication
(10) Pub. No.: US 2005/0027260 A1
(43) Pub. Date: Feb. 3, 2005

(54) Safety syringe

(76) Inventor: Hsien Tsung Wang, Taichung (TW)

Correspondence Address: Bacon & Thomas, 4th Floor, 625 Slaters Lane, Alexandria, VA, 22314-1176, US

(21) Appl. No.: 10/478,309

(22) PCT Filed: Jun. 5, 2002
(86) PCT No.: PCT/US02/16567

(30) Foreign Application Priority Data
Jun. 5, 2001 (CN) 01226524.1

(51) Int. Cl. A61M 5/32

(52) U.S. Cl. 604/198

(57) ABSTRACT

A safety syringe comprises a sleeve; a hollow barrel including a front nose; a needle; and a plunger snugly fitted within and slide through the barrel. After use, pull the plunger to cause the barrel to slide rearward until the needle is received in the sleeve again, thereby preventing a medical worker from being contaminated by microorganisms or blood on needle by accidentally pricking.

### BACKGROUND OF THE INVENTION

1. Field of the Invention

The present invention relates to the field of hypodermic needles and more particularly to an improved syringe with enhanced safety characteristics.

2. Description of Related Art

A conventional syringe is shown in FIG. 10 comprising a hollow syringe barrel 1, a needle 2, and a cap 3. In use, remove cap 3 from needle 2 prior to dispensing fluid from barrel 1. After use, put cap 3 onto needle 2 again. Since putting cap 3 onto needle 2 is done in front of needle 2, however, it is possible of accidentally pricking the medical worker by the tip 2a of needle 2 and thereby contaminating the medical worker with microorganisms or blood on the needle 2. Thus, it is desirable to provide an improved safety syringe in order to overcome the above drawback of prior art.

### SUMMARY OF THE INVENTION

It is an object of the present invention to provide a safety syringe wherein the retraction of a needle into a syringe barrel is done in the rear of the needle after use, thereby preventing a medical worker from being contaminated by microorganisms or blood on the needle by accidentally pricking.

It is another object of the present invention to provide a retractable syringe comprising a cap; a sleeve including a front opening; a hollow barrel including a front nose; a needle unit including a needle housing sleeved on and attached to the nose of the barrel and a needle; and a plunger having a front plug sized to be snugly fitted within and slide through the barrel; wherein in use remove the cap from the sleeve, and dispense fluid from the barrel by pressing the plunger for causing the needle to extend from the opening of the sleeve; and after use pull the plunger to cause the barrel to slide rearward until the needle is received in the sleeve again, and put the cap onto the sleeve.

In one aspect of the present invention, the sleeve further comprises a plurality of opposite flexible raised portions on an inner surface near a rear end, and the barrel further comprises a plurality of grooves on a surface with the raised portions slidably defined therein.

In another aspect of the present invention, each groove comprises a rear end, a turning point, a transverse section, and a front end. In still another aspect of the present invention, the barrel further comprises a plurality of opposite flexible flanges on the surface in a rear end.

In still another aspect of the present invention, the barrel further comprises an internal chamber sized to be snugly fitted onto an adapter of one of an intravenous (IV) infusion set, means for blood donation, and means for hypodermic injection.

In still another aspect of the present invention, the barrel further comprises a peripheral recess near the nose, and the sleeve further comprises a plurality of opposite flexible raised portions on an inner surface near a rear end secured in the recess in an unused state.

It is a further object of the present invention to provide a retractable syringe comprising a cap; a sleeve including a forward nose, an opening in a front end of the nose, a plurality of opposite slits in a rear end, and a plurality of opposite flexible raised portions on an inner surface near the rear end; a hollow barrel including a peripheral recess near a rear end for receiving the raised portions in an unused state, a front opening, and a rear opening; a needle unit including a needle housing sleeved on and attached to the nose of the barrel and a needle; and a plunger having a front plug sized to be snugly fitted within and slide through the barrel; wherein in use remove the cap from the sleeve, press the plunger to disengage the raised portions from the recess, and press the plunger for causing the needle to extend from the opening of the sleeve; and after use pull the plunger to cause the barrel to slide rearward until the needle is received in the sleeve again, and put the cap onto the sleeve.

In still another aspect of the present invention, the number of the slits on opposite side are two and that of the raised portions are two.

The above and other objects, features and advantages of the present invention will become apparent from the following detailed description taken with the accompanying drawings.

### BRIEF DESCRIPTION OF THE DRAWINGS

FIG. 1 is a perspective view of a first preferred embodiment of safety syringe according to the invention;

FIG. 2A is an exploded perspective view of FIG. 1;

FIG. 2B is similar to FIG. 2A where a second embodiment of sleeve member and syringe barrel is shown;

FIG. 2C is an enlarged fragmentary view of the sleeve member of FIG. 2B;

FIG. 2D is cross-sectional view of the sleeve member of FIG. 2B;

FIG. 3 is a cross-sectional view of FIG. 1 in an unused state;

FIG. 3A is a view similar to FIG. 3 where the syringe is in a use state with cap removed;

FIG. 4 is a side view in part section of a second preferred embodiment of safety syringe according to the invention;

FIG. 5 is a side view in part section of a third preferred embodiment of safety syringe according to the invention;

FIG. 6 is a side view in part section of a fourth preferred embodiment of safety syringe according to the invention;

FIG. 7 is an exploded view of a fifth preferred embodiment of safety syringe according to the invention;

FIG. 8 is a perspective view of a sixth preferred embodiment of safety syringe according to the invention;

FIG. 8A is an exploded perspective view of FIG. 8;

FIG. 9 is a side view in part section of FIG. 8 in an unused state;

FIG. 9A is a view similar to FIG. 9 where the syringe is in a use state with cap removed; and

FIG. 10 is a perspective view of a conventional syringe in a use state.

### DETAILED DESCRIPTION OF THE PREFERRED EMBODIMENTS

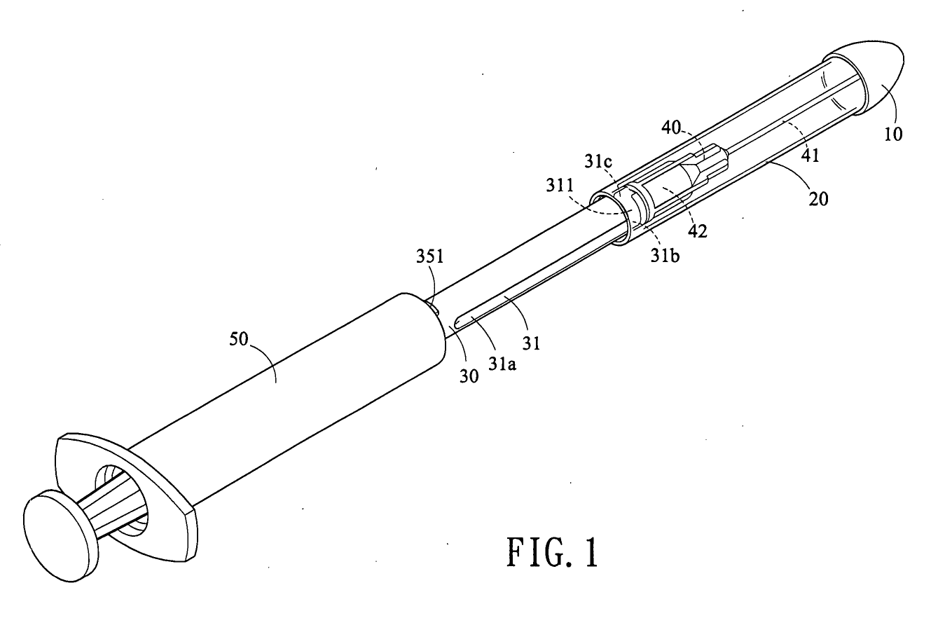

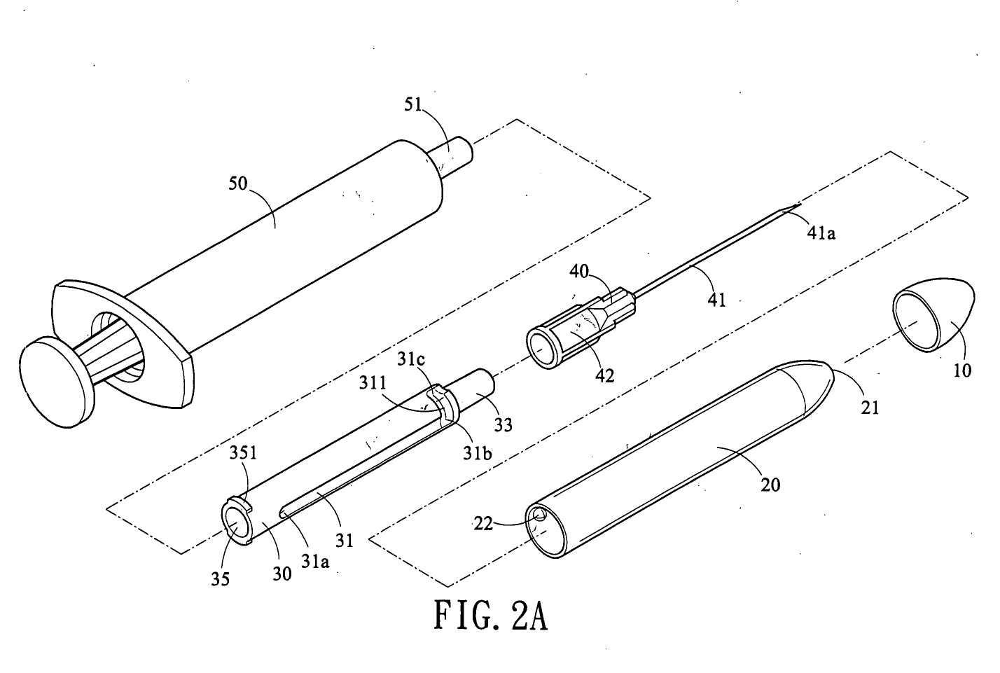

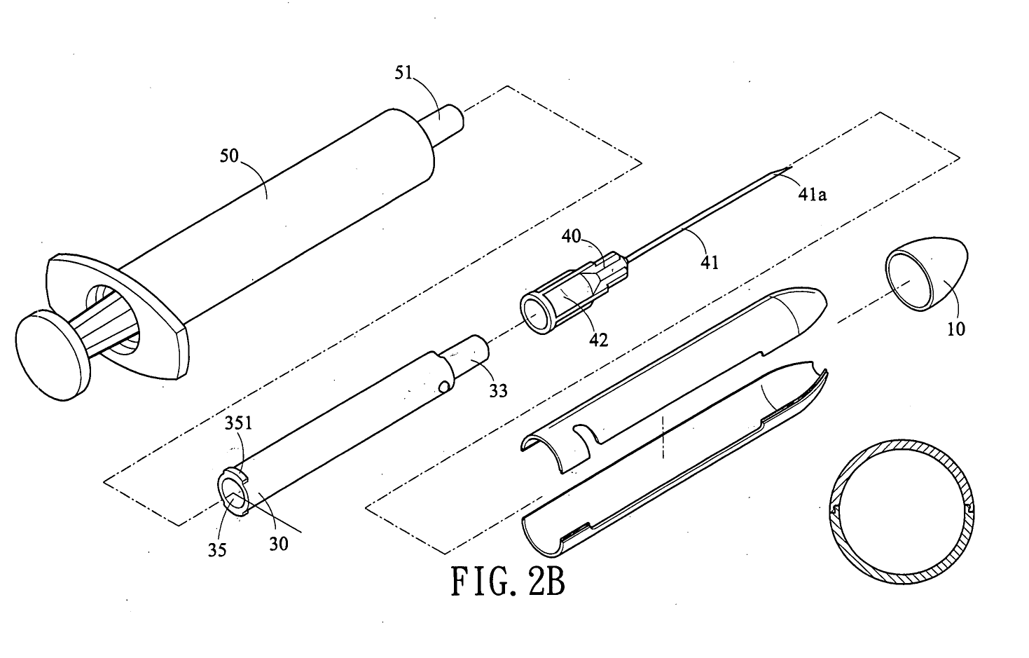

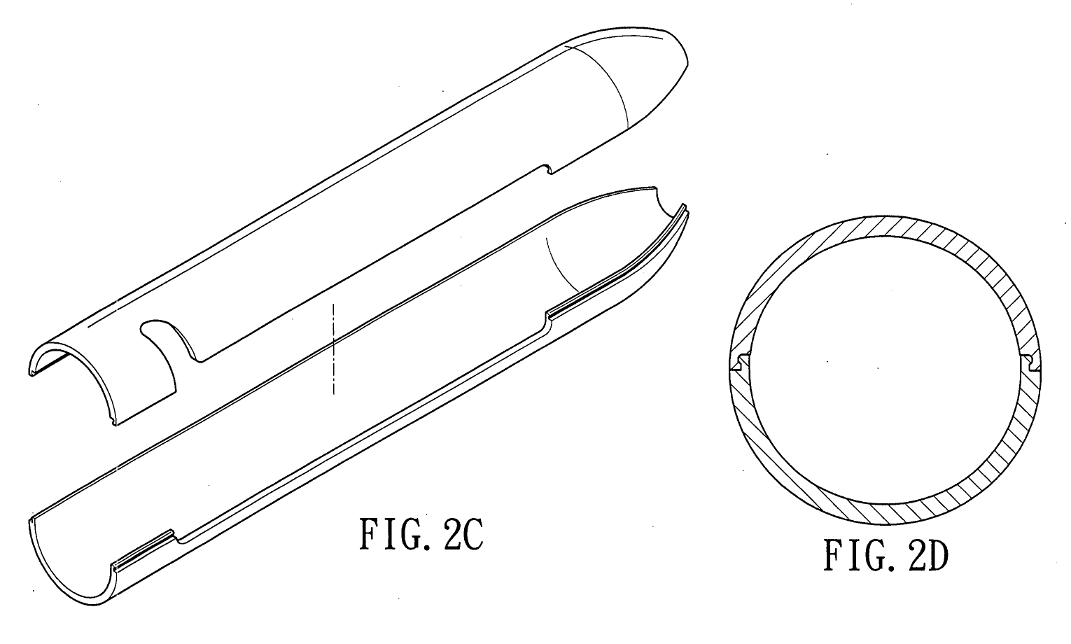

Referring to FIGS. 1 to 2A, there is shown a first preferred embodiment of safety syringe constructed in accordance with the invention. The syringe comprises a cup-like cap 10; a substantially cylindrical sleeve member 20 including an opening 21 in the front tapered end and a pair of opposite flexible raised portions 22 on the inner surface near the rear end; a hollow syringe barrel 30 including a front nose 33, a pair of opposite grooves 31 on the surface with raised portions 22 slidably defined therein, each groove 31 including a rear end 31a, a turning point 31b, a transverse section 311, and a front end 31c, an internal chamber 35, and a pair of opposite flexible flanges 351 on the surface in the rear end; a needle unit 40 including a needle housing 42 sleeved on and attached to nose 33 and a needle 41 having a tip 41a; and a plunger 50 having a front plug 51 sized to be snugly fitted within and slide through the internal chamber 35 and a forward end having a mating mechanism (not shown) releasably coupled to flanges 351.

Referring to FIGS. 2B to 2D, a second embodiment of sleeve member 20 and syringe barrel 30 is shown. The differences between first and second embodiments of sleeve member 20 and syringe barrel 30 will now be described. The members of syringe barrel 30 such as grooves 31 are replaced by a raised portion 32 near nose 33. Also, raised portions 22 are eliminated in sleeve member 20. In addition, sleeve member 20 consists of two mating portions 20A and 20B. Upper sleeve member 20B comprises two spaced latched edges 23B on the bottom, a recessed portion between latched edges 23B, and a slot 22C proximate the rear latched edge 23B. Lower sleeve member 20A comprises two spaced mating latched edges 23A on the top and a recessed portion between latched edges 23A combined with the recessed portion of upper sleeve member 20B to form a slit in communication with slot 22C wherein raised portion 32 may slide in a defined path in the slit and slot 22C during the fluid injection or needle 41 retraction operation of the syringe. A claw 231 is formed on each latched edge 23B and a mating claw 232 is formed on each latched edge 23A. Hence, a complete sleeve member 20 is formed by snapping claws 231 of upper sleeve member 20B onto mating claws 232 of lower sleeve member 20A (FIG. 2D).

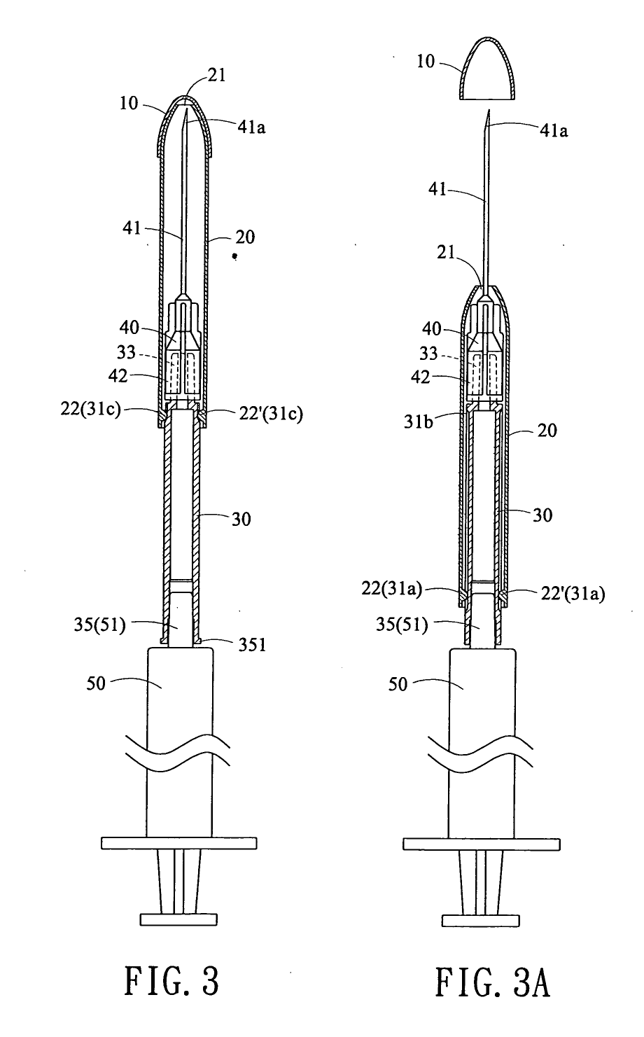

Referring to FIGS. 3 and 3A an operation of the first preferred embodiment of safety syringe is illustrated. In use, first remove cap 10 from sleeve member 20. Then dispense fluid (for hypodermic injection purpose) from barrel 30 completely by pressing handle of plunger 50. During the dispensing, tip 41a of needle 41 gradually extends from opening 21 by sliding the raised portions 22 from front ends 31c to rear ends 31a through transverse sections 311 and turning points 31b. After use, simply pulling handle of plunger 50 to cause the raised portions 22 to slide reversely, i.e., from rear ends 31a to front ends 31c through turning points 31b and transverse sections 311. During the pulling of handle of plunger 50, tip 41a of needle 41 gradually retracts into sleeve member 20 until in place. Finally, put cap 10 onto sleeve member 20 again. It is important to note that since the retraction of needle 41 into syringe barrel 30 is done, a medical worker's hand is always positioned in the rear of the needle 41 after use, thereby preventing a medical worker from being contaminated by microorganisms or blood on the needle 41 by accidentally pricking.

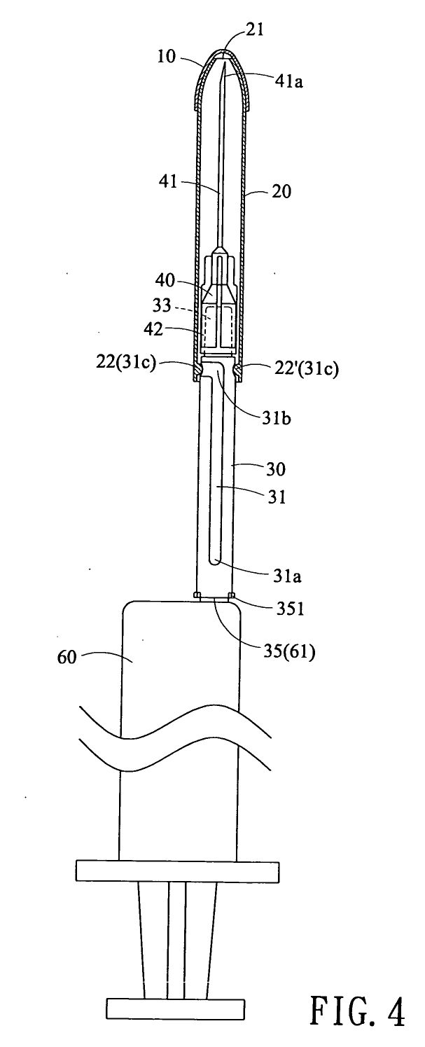

Referring to FIG. 4, there is shown a second preferred embodiment of safety syringe according to the invention. The difference of this and first embodiments will now be described. The plunger 50 is replaced by a larger plunger 60 adapted for blood donation usage. Plunger 60 has a front plug 61 sized to be snugly fitted within and slide through the internal chamber 35 and a forward end having a mating mechanism (not shown) releasably coupled to flanges 351.

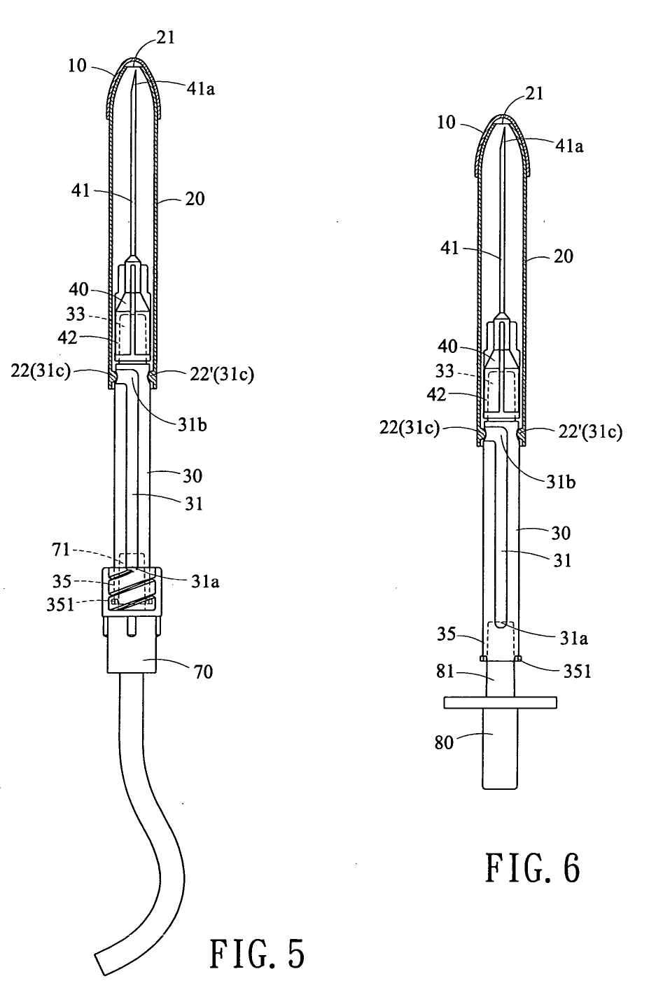

Referring to FIG. 5, there is shown a third preferred embodiment of safety syringe according to the invention. The difference of this and first embodiments will now be described. The plunger 50 is replaced by an adapter 70 of an intravenous (IV) infusion set. The adapter 70 has a front plug 71 sized to be snugly fitted within and slide through the internal chamber 35 and a forward end having a mating mechanism (not shown) releasably coupled to flanges 351.

Referring to FIG. 6, there is shown a fourth preferred embodiment of safety syringe according to the invention. The difference of this and first embodiments will now be described. The plunger 50 is replaced by an adapter 80 of another type of IV infusion set. The adapter 80 has a front plug 81 sized to be snugly fitted within and slide through the internal chamber 35 and an enlarged forward end having a mating mechanism (not shown) releasably coupled to flanges 351.

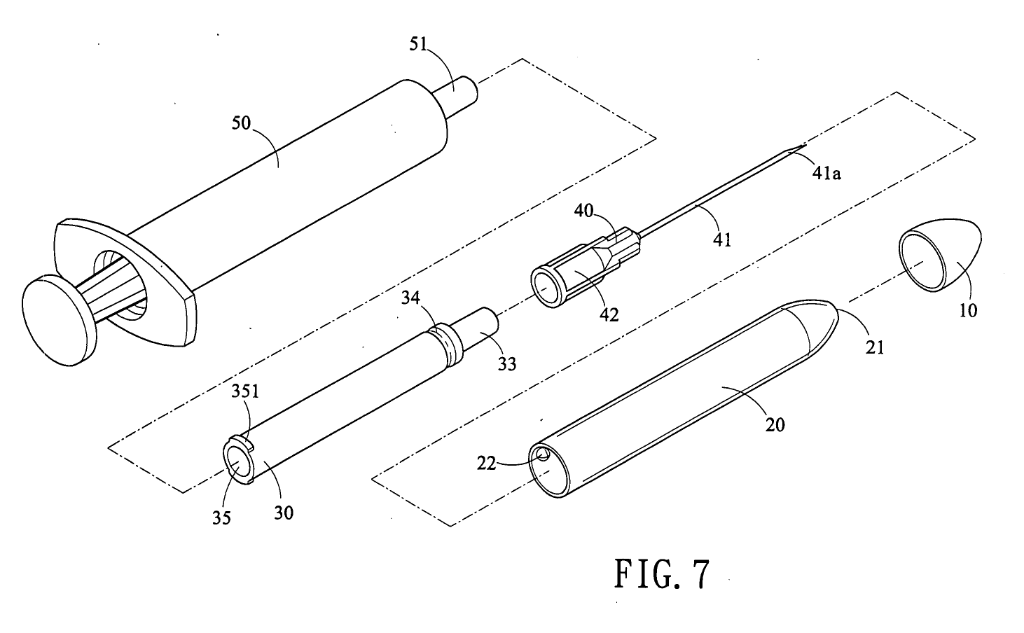

Referring to FIG. 7, there is shown a fifth preferred embodiment of safety syringe according to the invention. The difference of this and first embodiments will now be described. The members of syringe barrel 30 such as opposite grooves 31 are replaced by a peripheral recess 34 near nose 33. In an unused state, raised portions 22 are secured in recess 34. To the contrary user may clear raised portions 22 from recess 34 and slide down sleeve member 20 for receiving the syringe barrel 30 until in place.

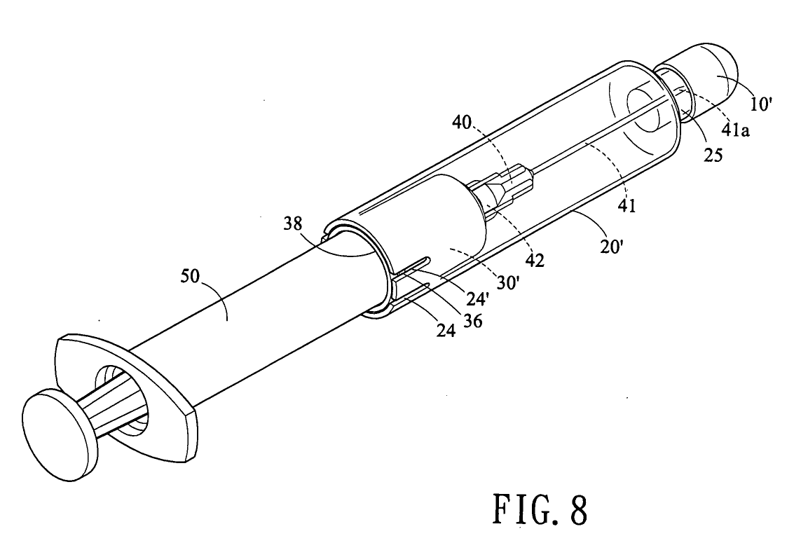

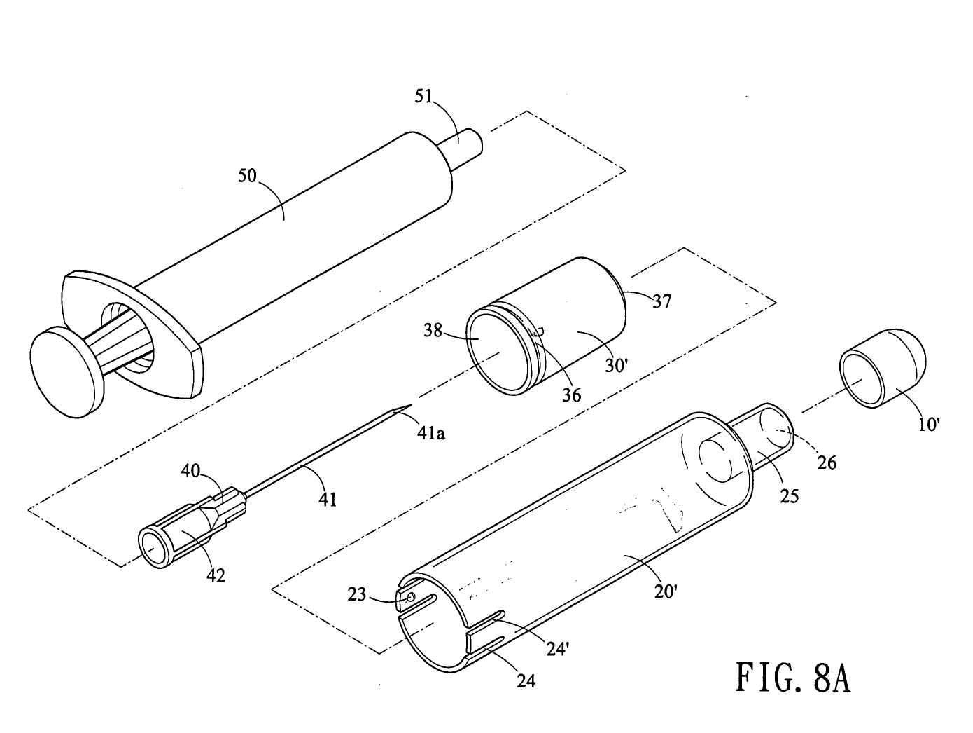

Referring to FIGS. 8 and 8A, there is shown a sixth preferred embodiment of safety syringe according to the invention. The difference of this and first embodiments will now be described. The sleeve member 20 is replaced by a sleeve member 20′ including a forward nose 25, an opening 26 in the front end of nose 25, two sets of a plurality of opposite slits 24 and 24′ (each set having two slits as shown) in the rear end, and a pair of opposite flexible raised portions 23 on the inner surface near the rear end (i.e., each raised portion 23 is on the inner surface of a portion between two slits 24 and 24′ for allowing the portion between slits 24 and 24′ to be flexible transversely for conforming the engagement/disengagement of recess 36 and raised portions 23). Further, the syringe barrel 30 is replaced by a substantially cylindrical barrel 30′ including a peripheral recess 36 near the rear end for receiving raised portions 23 in an unused state, a front opening 37, and a rear opening 38.

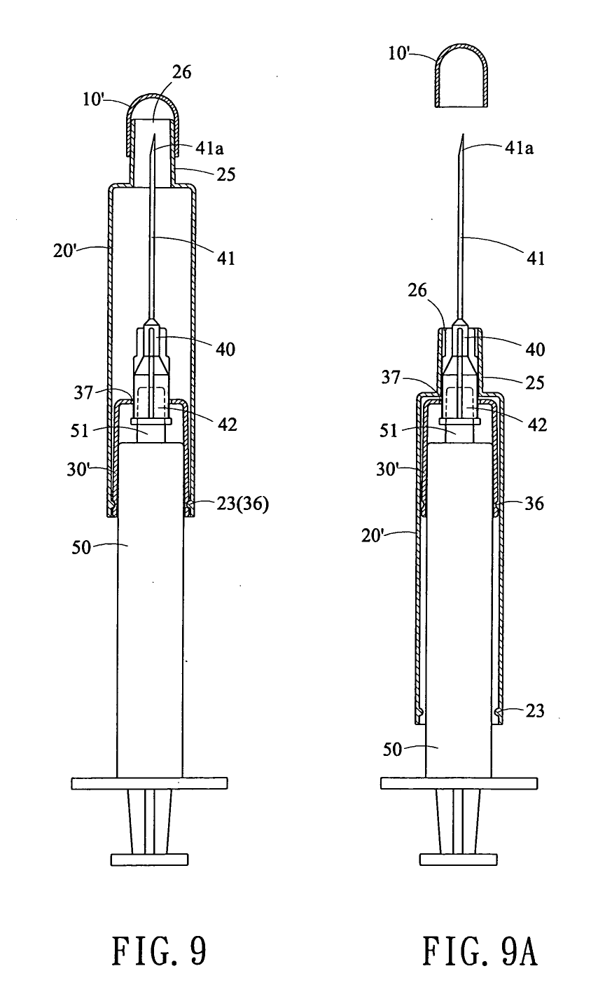

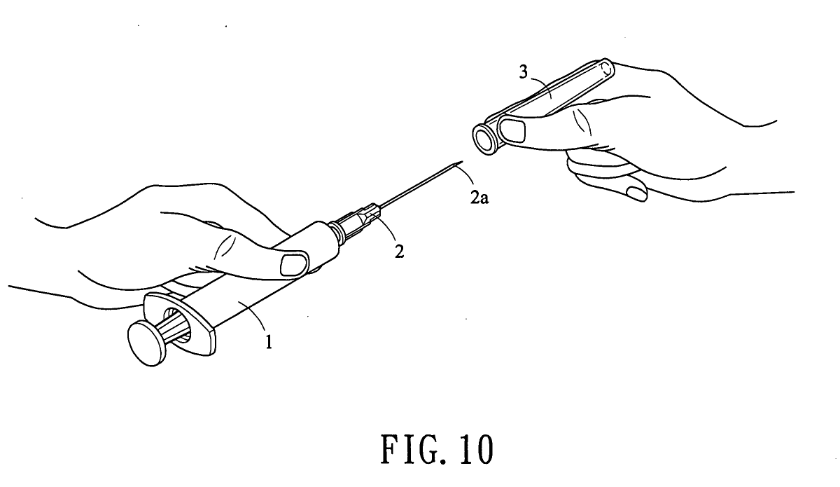

Referring to FIGS. 9 and 9A, the operation of the sixth embodiment is substantially the same as that of the first one. In detail, in use first remove cap 10′ from nose 25. Then press handle of plunger 50 to disengage raised portions 23 from recess 36 prior to dispensing fluid from barrel 30′ completely. During the dispensing, tip 41a of needle 41 gradually extends from sleeve member 20 until the front end of barrel 30′ engages with the inner surface of the shoulder portion with nose 25 extended therefrom. After use, simply pulling handle of plunger 50 to cause the raised portions 23 to slide reversely until raised portions 23 engages with recess 36 again.

The benefit of this invention includes the retraction of needle 41 into syringe barrel 30 being done in the rear of the needle 41 after use, thereby preventing a medical worker from being contaminated by microorganisms or blood on the needle 41 by accidentally pricking.

While the invention herein disclosed has been described by means of specific embodiments, numerous modifications and variations could be made thereto by those skilled in the art without departing from the scope and spirit of the invention set forth in the claims.

1. A retractable syringe comprising:
a cap;
a sleeve including a front opening;
a hollow barrel including a front nose;
a needle unit including a needle housing sleeved on and attached to said nose of said barrel and a needle; and
a plunger having a front plug sized to be snugly fitted within and slide through said barrel;
wherein in use remove said cap from said sleeve, and dispense fluid from said barrel by pressing said plunger for causing said needle to extend from said opening of said sleeve; and after use pull said plunger to cause said barrel to slide rearward until said needle is received in said sleeve again, and put said cap onto said sleeve.

2. The safety syringe of claim 1, wherein said sleeve further comprises a plurality of opposite flexible raised portions on an inner surface near a rear end, and said barrel further comprises a plurality of grooves on a surface with said raised portions slidably defined therein.

3. The safety syringe of claim 2, wherein each groove comprises a rear end, a turning point, a transverse section, and a front end.

4. The safety syringe of claim 1, wherein said barrel further comprises a plurality of opposite flexible flanges on the surface in a rear end.

5. The safety syringe of claim 1, wherein said barrel further comprises an internal chamber sized to be snugly fitted onto an adapter of one of an intravenous (IV) infusion set, means for blood donation, and means for hypodermic injection.

6. The safety syringe of claim 1, wherein said barrel further comprises a peripheral recess near said nose, and said sleeve further comprises a plurality of opposite flexible raised portions on an inner surface near a rear end secured in said recess in an unused state.

7. A retractable syringe comprising:
a cap;
a sleeve including a forward nose, an opening in a front end of said nose, a plurality of opposite slits in a rear end, and a plurality of opposite flexible raised portions on an inner surface near the rear end;
a hollow barrel including a peripheral recess near a rear end for receiving said raised portions in an unused state, a front opening, and a rear opening;
a needle unit including a needle housing sleeved on and attached to said nose of said barrel and a needle; and
a plunger having a front plug sized to be snugly fitted within and slide through said barrel;
wherein in use remove said cap from said sleeve, press said plunger to disengage said raised portions from said recess, and press said plunger for causing said needle to extend from said opening of said sleeve; and after use pull said plunger to cause said barrel to slide rearward until said needle is received in said sleeve again, and put said cap onto said sleeve.

8. The safety syringe of claim 7, wherein the number of said slits on either side are two and that of said raised portions are two.
